(19) United States
(12) Patent Application Publication
(10) Pub. No.: US 2014/0093573 A1
(43) Pub. Date: Apr. 3, 2014

(54) NON-ENTERIC COATED PHARMACEUTICAL COMPOSITION AND USE THEREOF

(71) Applicant: NATIONAL TAIWAN UNIVERSITY, Taipei (TW)

(72) Inventors: Wen Jen LIN, Taipei City (TW); Milind Alai, Taipei City (TW)

(73) Assignee: National Taiwan University, Taipei (TW)

(21) Appl. No.: 13/630,814

(22) Filed: Sep. 28, 2012

(51) Int. Cl. A61K 9/14; A61P 1/00; A61P 1/04; A61K 9/00; A61K 31/4439; B82Y 5/00

(52) U.S. Cl. 424/490; 424/400; 514/338; 977/773

(57) ABSTRACT

A non-enteric coated pharmaceutical composition having an enhanced bioavailability comprising an acid-labile active ingredient and a nanolized biocompatible polymer, wherein the acid-labile active ingredient is mixed with and trapped by the nanolized biocompatible polymer, and the acid-labile active ingredient is sustainably released from the nanolized biocompatible polymer.

### BACKGROUND

1. Field of the Invention

The present disclosure relates to a pharmaceutical composition, and more particularly to a non-enteric coated pharmaceutical composition.

2. Description of the Related Art

Acid related disorders such as peptic ulcer and gastro-esophageal reflux disease (GERD) frequently occur in older people and are associated with morbidity. Currently, acid suppression is the primary goal of treatment of acid related disorders. Proton pump inhibitors (PPIs) are most effective for ulcer healing and greater symptomatic relief in patients with acid related disorders. Proton pump inhibitors may be enteric coated—coated with a material that permits transit through the stomach to the small intestine before the medication is released. The action mechanism of proton pump inhibitors is conducted via inhibiting H⁺/K⁺ ATPase (also known as a proton pump), an enzyme present in the gastric parietal cells, to prohibit gastric acid secretion. It provides earlier and better symptom relief than the other PPI. Conventionally, a single dose of PPI per day is used to control gastric acid secretion. However, some patients experience a nighttime (nocturnal) acid breakthrough event where the secretory activity of proton pump returns. Therefore, there is a need for a sustainably-released dosage form containing PPI that can reliably provide long-term stomach-specific acid suppression in order to prevent the recurrence of gastro-esophageal reflux disease, while being administered on a once daily basis.

Nanoparticles may be used in the delivery of drugs, as small particles may be efficiently taken up by macrophages, mainly by phagocytosis.

Until now, no non-enteric coated and stomach-specific nanoparticulate dosage forms comprised of PPIs have been developed.

### SUMMARY

The present disclosure describes a non-enteric coated pharmaceutical composition having an enhanced bioavailability comprising an acid-labile active to ingredient and a nanolized biocompatible polymer, wherein the acid-labile active ingredient is mixed with and trapped by the nanolized biocompatible polymer, and the acid-labile active ingredient is sustainably released from the nanolized biocompatible polymer.

The present disclosure also provides a method for treating or preventing a stomach disorder by administering a non-enteric coated pharmaceutical composition to a patient subjected to said stomach disorder, and wherein the non-enteric coated pharmaceutical composition comprises an acid-labile active ingredient for treating or preventing said stomach disorder and a nanolized biocompatible polymer for mixing and trapping the acid-labile active ingredient.

In the present disclosure, “non-enteric coated” refers to dosage forms without an enteric coating film.

### BRIEF DESCRIPTION OF THE DRAWINGS

FIG. 1 depicts TEM images of (A) ERSNPs-LPZ and (B) PLGANPs-LPZ, in accordance with an embodiment of the present disclosure;

FIG. 2 depicts release profiles of LPZ from ERSNPs-LPZ and PLGANPs-LPZ in pH 7.4 phosphate buffer solution (n=3), in accordance with an embodiment of the present disclosure;

FIG. 3 depicts (A) flow cytometry images and (B) cellular uptake efficiency (%) of (a) control (HBSS), (b) coumarin-6, (c) ERSNPs-C6 and (d) PLGANPs-C6 in Caco-2 cells after 0.5 h incubation (n=3), in accordance with an embodiment of the present disclosure;

FIG. 4 depicts confocal microscopic images of Caco-2 cells after 0.5 hours of incubation at 37° C. with ERSNPs-C6 and PLGANPs-C6, in accordance with an embodiment of the present disclosure;

FIG. 5 depicts fluorescence microscopic images of sectioned stomach tissues: (A) and (B) after H/E stain; (C) and (D) after oral administration of ERSNPs-C6-NaHCO₃ (100 mg/kg) for 4 h; (E) and (F) after oral administration of PLGANPs-C6-NaHCO₃ (100 mg/kg) for 4 h, in accordance with an embodiment of the present disclosure;

FIG. 6 depicts distribution of nanoparticles in ulcerated and non-ulcerated stomach tissues of rats after oral administrations of ERSNPs-C6-NaHCO₃ and PLGANPs-C6-NaHCO₃ (100 mg/kg) for 4 h (n=4), in accordance with an embodiment of the present disclosure;

FIG. 7 depicts plasma LPZ concentration versus time profiles in ulcer induced male Wistar rats after oral administrations of a known commercial product RICH® (a capsule containing enteric coated pellets) or the nanoparticulate dosage form of the present disclosure ERSNPs-LPZ-NaHCO₃ and PLGANPs-LPZ-NaHCO₃ (5 mg LPZ/kg) (n=4), in accordance with an embodiment of the present disclosure; and

FIG. 8 depicts (A) the photographic images of stomachs in ulcer induced rats after oral administrations of (a) saline solution (control), (b) ERSNPs-LPZ-NaHCO₃ and (c) PLGANPs-LPZ-NaHCO₃ (5 mg LPZ/kg/day) for 7 days, wherein the arrows indicate the ulcerated regions; and (B) the calculated gastric ulcer indexes after 7-day treatment, in accordance with an embodiment of the present disclosure.

### DETAILED DESCRIPTION

The present disclosure provides a non-enteric coated pharmaceutical composition having an enhanced bioavailability comprising an acid-labile active ingredient and a nanolized biocompatible polymer, wherein the acid-labile active ingredient is mixed with and trapped by the nanolized biocompatible polymer, and the acid-labile active ingredient is sustainably released from the nanolized biocompatible polymer.

The non-enteric coated pharmaceutical composition may be in a form of nanoparticle, i.e. a particle with a diameter less than 1 micrometer. In some embodiments, the nanoparticle has an average diameter of about 100 nm to about 950 nm such as, without limitation, 120 m, 150 nm, 200 nm, 250 nm, 300 nm, 350 nm, 500 nm, 700 nm, 850 nm, or any value between the above two points. In a preferred embodiment, the nanoparticle has an average diameter of about 120 nm to about 300 nm, and more preferably has a polydispersity index less than about 0.3. In a preferred embodiment, the polydispersity index is less than 0.25, such as 0.2 or 0.15.

In an embodiment, the active ingredient contained in the non-enteric coated pharmaceutical composition may be an acid-labile drug, namely, a drug that is unstable or easily destroyed in an acid environment. The acid-labile drug includes, without limitation, omeprazole, lansoprazole, dexlansoprazole, esomeprazole, pantoprazole, minoprazole, pantoprazole and rabeprazole, penicillin salts, bacitracin, aureomycin, cephalosporins, chloromycetin, erythromycin, dihydrostreptomycin, streptomycin, novobiocin, polymyxin, subtilin, famotidine, progabide, clorazepate, deramciclane, pravastatin, milameline, digitalis glycosides, etoposide, quinapril, quinoxaline-2-carboxylic acid, sulphanilamide, beta carotene, cladribine, didanosine, amylase, lipase, protease, adrenalin, insulin, heparin, estrogens, cisapride, ranitidine, pancreatin, cimetidine, and the like.

In an embodiment, the pharmaceutical composition comprises a stomach-specific dosage form. In an embodiment, the active ingredient contained in the non-enteric coated pharmaceutical composition may be used for treating or preventing stomach disorder involving abnormal gastric acid secretion, such as peptic ulcer, gastro-esophageal reflux disease (GERD), and the like. The active ingredient can be antacids, H2 receptor antagonists, proton pump inhibitors, or any combination thereof. In a preferred embodiment, the active ingredient is a proton pump inhibitor, which can be selected from omeprazole, lansoprazole, dexlansoprazole, esomeprazole, pantoprazole, minoprazole, pantoprazole, rabeprazole, or any combination thereof. In a preferred embodiment, the active ingredient may be lansoprazole.

To enhance the therapy efficiency of the drug in situ, for example, in the ulcer site of stomach, the conventional enteric coating layer is forsaken, and a nanolized biocompatible polymer is applied to mix with and trap the acid-labile active ingredient in order to form nanoparticles. The nanoparticles are able to attach in the stomach via attachment between the nanolized biocompatible polymer and the gastric wall, and then the nanoparticles may sustainably release the acid-labile active ingredient to in situ, thereby avoiding an initial burst release of the active ingredient.

In the present disclosure, the polymer contained in the non-enteric coated pharmaceutical composition may not be limited so long as the polymer is biocompatible or biodegradable, and able to be nanolized. In some embodiments, the biocompatible polymer can be selected from the biocompatible polymer is selected a group consisting of poly(acrylic acid), polyacrylate, polycyanoacrylate, polyanhydride, polyamide, polyester, poly(orthoester), polyesteramide, polydihydropyran, poly(lactic acid), poly(glycolic acid), poly(lactic-co-glycolic acid), poly(ethylene glycol), polyvinyl alcohol (PVA), poly(sulfobetaine methacrylate) (PSBMA), polyhydroxyalkanoate (PHA), poly(hydroxyhexanoate), polyphosphazene, polypeptide, or any combination thereof.

In a preferred embodiment, the biocompatible polymer is a copolymer having monomers selected from a group consisting of ethyl acrylate, methyl methacrylate, and methacrylic acid. In an embodiment, the biocompatible polymer is Eudragit RS100 polymer, which is a non-biodegradable but biocompatible mucoadhesive polymer.

In another preferred embodiment, the biocompatible polymer is poly(lactic-co-glycolic acid) (PLGA).

The present disclosure also provides a method for treating or preventing stomach disorders, such as peptic ulcer, gastro-esophageal reflux disease (GERD), and the like, comprising administering a non-enteric coated pharmaceutical composition to a patient subjected to said stomach disorder, wherein the non-enteric coated pharmaceutical composition comprises an acid-labile active ingredient for treating or preventing said stomach disorder and a nanolized biocompatible polymer for mixing and trapping the acid-labile active ingredient.

In an embodiment, the pharmaceutical composition is a nanoparticle. In an embodiment, the pharmaceutical composition further comprises a capsule for carry the nanoparticles.

In an embodiment, the active ingredient for treating or preventing stomach disorders involving abnormal gastric acid secretion includes antacids, H2 receptor antagonists, or proton pump inhibitors such as omeprazole, lansoprazole, dexlansoprazole, esomeprazole, pantoprazole, minoprazole, pantoprazole, rabeprazole and the like. In an embodiment, the biocompatible polymer is PLGA, or a copolymer having monomers selected from a group consisting of ethyl acrylate, methyl methacrylate, and methacrylic acid.

In the present method, the pharmaceutical composition is a sustain release form having a controlled release of the active ingredient for about 24 hours per dosage. Further, the pharmaceutical composition has enhanced bioavailability compared with the conventional enteric-coated formulation. Accordingly, the pharmaceutical composition can be provided to the patient in an administrative frequency of about once daily. The dosage can be determined by a skilled person depending on the conditions of the patient, type of disease, severity of disease, and the like.

Examples of non-enteric coated pharmaceutical compositions are further described hereafter.

### EXAMPLES

Materials

LPZ was obtained from Alcon Biosciences Private Ltd. (Mumbai, India). The polymers used in this study were Eudragit® RS100 (Degussa, Darmstadt, Germany) and poly (lactic-co-glycolic acid) (PLGA, M_{w} 28000 Da, copolymer ratio 50:50, Boehringer Ingelheim, Ingelheim, Germany). Coumarin-6 (Sigma-Aldrich, St. Louis, USA) was used as a fluorescence marker. Sodium bicarbonate (NaHCO₃) was purchased from Sigma-Aldrich (Atlanta, USA). Acetone, acetonitrile and methanol were of HPLC grade. All other chemicals and solvents were of reagent grade. Human colon adenocarcinoma cell line, Caco-2, was a gift from Dr. Li-Juan Shen, Graduate Institute of Pharmaceutical Sciences, National Taiwan University, Taipei, Taiwan, and originated from the American Type Culture Collection (ATCC, Manassas, USA). Dulbecco Modified Eagle's Medium (DMEM) (with 4.5 g/L D-glucose, with L-glutamine, without sodium pyruvate and sodium bicarbonate), non-essential amino acids (NEAA) and mycoplasma tested fetal bovine serum (FBS) were purchased from Biological Industries (Beit-Haemek, Israel). Penicillin-streptomycin, trypsin-EDTA consist of 0.5% w/v trypsin in PBS, sodium pyruvate, Hank's balanced salt solution buffer (HBSS), propidium iodide (PI), ribonuclease A (RNase A) were purchased from Invitrogen Corporation (Carlsbad, USA). The 6-well tissue culture plates were purchased from Becton Dickinson Labware (NJ, USA). The hanging cell culture inserts for 6 well plates (Millicell®, polyethylene terephthalate, 1 μm pore size, 4.5 cm² membrane area) were purchased from Millipore Corporation (NJ, USA).

### Example 1

### Preparation of LPZ-Loaded Nanoparticles

### Example 1.1

### Preparation of ERSNPs-LPZ

LPZ-loaded Eudragit RS 100 nanoparticles (ERSNPs-LPZ) were prepared by an oil-in-water (o/w) emulsion solvent evaporation method. Eudragit® RS100 (200 mg) and LPZ (200 mg) were dissolved in 10 mL dichloromethane/methanol mixture (5/5, v/v). The organic phase was added into 100 mL aqueous PVA solution (0.25% w/v, pH 9.0) under sonication using an ultrasonic probe (Sonics and Materials Inc., Newtown, USA) set at 50 W of energy output with a pulse mode (pulse on 30 s, pulse off 10 s) at 4° C. for 20 min. The organic solvent was evaporated by magnetic stirring at room temperature for 3 hours followed by using rotarvapor under reduced pressure at 35° C. for 5 min. The nanoparticles were recovered after centrifugation at 17,000 rpm for 30 min (Avanti J26 XP centrifuge, Beckman Coulter, Miami, USA). The collected nanoparticles were washed with deionized water three times. Finally, the nanoparticles were resuspended in 1 mL deionized water containing 5% w/v glucose and freeze dried.

### Example 1.2

### Preparation of PLGANPs-LPZ

LPZ-loaded PLGA nanoparticles (PLGANPs-LPZ) were prepared by a water-in-oil-in-water (w/o/w) emulsion solvent evaporation method. PLGA (200 mg) and LPZ (100 mg) were dissolved in 10 mL dichloromethane/acetone mixture (5/5 v/v). An aqueous solution of NaHCO₃ (1 mL, 0.2%) was added to PLGA solution and emulsified to obtain a primary water-in-oil emulsion using an ultrasonic probe set at 50 W of energy output at 4° C. for 2 min. The primary emulsion was then added to 100 mL aqueous PVA solution (0.25% w/v, pH 9.0) and emulsified using an ultrasonic probe as mentioned in Example 1.1. The following preparation steps were the same as described for above Example 1.1.

### Example 2

### Characterization of Nanoparticles

The freeze dried nanoparticles were weighed and the yield was calculated as a percentage of the total amount of polymer and drug added initially by using Eq. (1).

$$Yield ( \% ) = \frac{Total amount of nanoparticles obtained ( mg )}{Total amount of drug and polymer added initially ( mg )} \times 100 \% \qquad ( 1 )$$

The particle size and zeta potential of nanoparticles were determined by Zetasizer (Nano ZS, Malvern Co. Ltd., Worcestershire, UK). The morphology of nanoparticles was examined by transmission electron microscope (TEM, H7100, Hitachi High-technologies Corporation, Tokyo, Japan). For LPZ content determination, about 5 mg of ERSNPs-LPZ and PLGANPs-LPZ were dissolved in 5 mL methanol and acetonitrile, respectively. Each sample was centrifuged at 14,000 rpm for 10 min and 20 μL aliquot of the supernatant were injected into HPLC. The HPLC system (Jasco International Company Ltd., Tokyo, Japan) was consisted of a pump (PU-2089) and a photo diode array detector (PDA, MD-2010) at 285 nm. A reversed phase silica column (C-18, 4.6×250 mm, 5 μm, Phenomenex Inc., USA) was used. The mobile phase was comprised by water, acetonitrile and triethylamine in the volume ratio of 50:50:0.1 (pH 7) with a flow rate of 1 mL/min The drug loading (DL) and encapsulation efficiency (EE) were calculated by Eq. (2) and Eq. (3).

$$\begin{matrix}DL ( \% ) = \frac{Determined amount of drug in nanoparticles}{Total amount of nanoparticles} \times 100 \% & ( 2 ) \\ EE ( \% ) = \frac{Determined amount of drug in nanoparticles}{Total amount of drug used for nanoparticles preparation} \times 100 \% & ( 3 )\end{matrix}$$

The HPLC analytical method was validated prior to sample analysis. It was linear over a concentration range of 5 to 200 μg/mL and the coefficients of determination (R²) were >0.9999. The accuracy was in the range of 94.00%-107.30%, and the precision expressed as the relative standard deviation was in the range of 0.13% to 5.49%.

Characterization of ERSNPs-LPZ

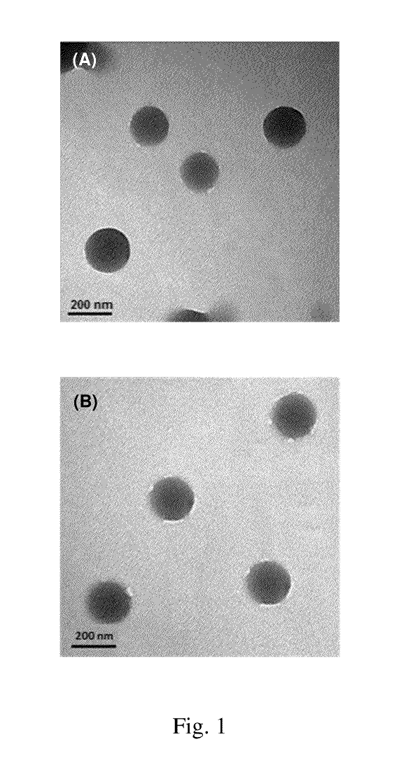

The yield of ERSNPs-LPZ was 78.29±2.09%. FIG. 1A depicts a TEM image of the ERSNPs-LPZ with spherical shape and smooth surface. The mean particle size was 203.9±4.9 nm with a polydispersity index 0.09±0.04 indicating a narrow size distribution. The ERSNPs-LPZ exhibited a zeta potential +38.5±0.3 mV. The drug loading and encapsulation efficiency of ERSNPs-LPZ were 43.67±0.54% and 79.28±0.94%, respectively.

Characterization of PLGANPs-LPZ

The yield of PLGANPs-LPZ was 75.34±3.56%. FIG. 1B depicts a TEM image of the PLGANPs-LPZ with spherical shape and smooth surface. The mean particle size was in the range of 219.2±2.9 nm with a polydispersity index 0.13±0.07 indicating the narrow size distribution. PLGANPs-LPZ exhibited a zeta potential −27.3±0.3 mV due to carboxylic groups of PLGA. The drug loading and encapsulation efficiency of PLGANPs-LPZ were 28.71±1.15% and 79.60±2.23%, respectively.

### Example 3

### In Vitro Drug Release

LPZ drug powder and nanoparticles equivalent to 1 mg LPZ were suspended in 5 mL pH 7.4 phosphate buffered solution which was placed in a dialysis bag (MWCO 6000-8000 Da). The dialysis bag was immersed in 100 mL of the same release medium and maintained at 37±0.5° C. in a shaker bath with a speed of 75 rpm. Samples (1 mL) were collected at time intervals of 0.5, 1, 2, 4, 6, 8, 12 and 24 hours, and the same volume of the fresh release medium was replaced. The amount of LPZ in each released sample was analyzed by HPLC method. The mathematical models were used to evaluate the release kinetics and mechanism of LPZ release from the nanoparticles.

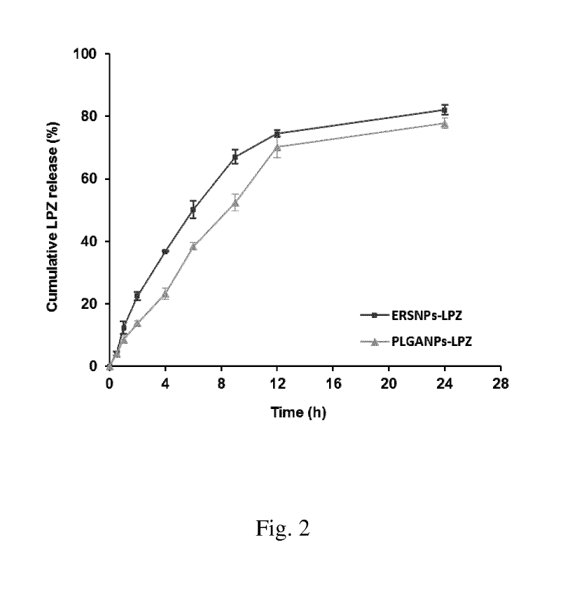

FIG. 2 depicts an in vitro release of ERSNPs-LPZ and PLGANPs-LPZ in pH 7.4 release medium. ERSNPs-LPZ and PLGANPs-LPZ showed sustained release patterns up to 24 hours. The in vitro release profiles (0-24 h) of ERSNPs-LPZ was best fitted by Higuchi's square root model, and the corresponding release rate constant was 19.77±0.13% h^{-1/2} with coefficient of determination (R²) 0.9418±0.011. The in vitro release profiles (0-24 h) of PLGANPs-LPZ was also fitted by Higuchi's square root model, and the corresponding release rate constant was 18.55±0.62% h^{-1/2} with coefficient of determination (R²) 0.9466±0.007. These suggested that the drug released from nanoparticles was dominated by diffusion mechanism.

### Example 4

### Preparation of Fluorescent Nanoparticles

### Example 4.1

### Preparation of Coumarin-6 Loaded Fluorescent Nanoparticles

Coumarin-6 loaded Eudragit® RS 100 nanoparticles (ERSNPs-C6) and PLGA nanoparticles (PLGANPs-C6) were prepared by o/w solvent evaporation method. Eudragit® RS100 or PLGA (200 mg) and 1 mg coumarin-6 were dissolved in 10 mL dichloromethane/acetone mixture (5/5 v/v). The following preparation steps were the same as described for above Example 1.1.

### Example 4.2

### Characterization of Fluorescent Nanoparticles

The amount of coumarin-6 entrapped in ERSNPs-C6 and PLGANPs-C6 was determined by fluorescence spectrophotometer (F4500, Hitachi Ltd., Tokyo, Japan). Nanoparticles (1 mg) were dissolved in 10 mL acetone and further diluted for fluorescence measurement at an excitation wavelength 430 nm and an emission wavelength 490 nm The fluorescence analytical method was validated prior to sample analysis. It was a linear over the concentration range of 5-150 ng/mL and the coefficients of determination (R²) were ≧0.9999. The accuracy was in the range of 99.00%-104.10% and the precision expressed as the relative standard deviation was in the range of 1.07%-8.25%.

The coumarin-6 loaded fluorescent nanoparticles were prepared to demonstrate the cellular uptake and biodistribution of positively charged ERSNPs-C6 and negatively charged PLGANPs-C6 nanoparticles. The mean particle sizes of ERSNPs-C6 and PLGANPs-C6 were 188.9±8.7 nm and 193.4±2.9 nm, and the corresponding zeta potentials were +39.4±0.6 mV and −24.5±0.7 mV. The dye to loadings of ERSNPs-C6 and PLGANPs-C6 were 0.35±0.03% and 0.088±0.003%, respectively.

### Example 4.3

### Cellular Uptake Study

Caco-2 cell monolayers approximately 21-24 days post seeding were used for the cellular uptake study. Before the experiments, the monolayers were washed with HBSS (pH 7.4) twice and then preincubated with HBSS at 37° C. for 30 min. The HBSS (control), free coumarin-6 solution (200 ng/mL), ERSNPs-C6 or PLGANPs-C6 suspension in HBSS equivalent to 200 ng/mL of coumarin-6 were added in the donor compartment while 3 mL of HBSS was added in the receiver compartment. These Caco-2 cell monolayers were incubated for 0.5 h and 1 h at 37° C. in an atmosphere of 5% CO₂ and 90% relative humidity. The flow cytometry and confocal microscopy were used to assess the intracellular fluorescence. After 0.5 h and 1 h of incubation, the cell monolayers were washed with phosphate buffered saline (PBS, pH 7.4) three times following by trypsinization for 5 min (100 μL, 0.25% trypsin EDTA). Trypsinization was stopped by adding 1 mL of cold PBS. Cells were detached from the inserts by pipetting and centrifuged at 1000 rpm for 5 min The cells were resuspended in 2 mL PBS and analyzed by using fluorescent activated flow cytometry (BD FACS Calibur) and BD CellQuest software (BD Biosciences, NJ, USA). This experiment was performed in triplicate.

For confocal microscopic study, after 0.5 hours of incubation at 37° C., cell monolayer was washed with PBS three times. The cell monolayers were then fixed with 3.7% paraformaldehyde solution in PBS for 30 min. The formaldehyde solution was removed after fixation and the cells were washed with PBS three times. The monolayers were treated with RNase solution (20 μg/mL) for 30 min, and the nuclei were stained with 4 μg/mL propidium iodide (PI) for 30 min. after being washed with PBS three times. The insert membrane with cell monolayer was removed from the hanging insert using a scalpel, then mounted on the glass slide with mounting medium Fluoromount™ (Sigma-Aldrich, St. Louis, USA), and covered. Images were captured using Leica confocal laser scanning microscopy imaging system (TCS SP5, Leica, Wetzlar, Germany).

Results of Cellular Uptake Test

The cellular uptake of coumarin-6 loaded fluorescent ERSNPs-C6 and PLGANPs-C6 in Caco-2 cell monolayer was monitored by flow cytometer and confocal microscope. The difference in intracellular fluorescence intensity as compared to HBSS incubated cells (control group) implied the uptake of fluorescent nanoparticles by Caco-2 cells.

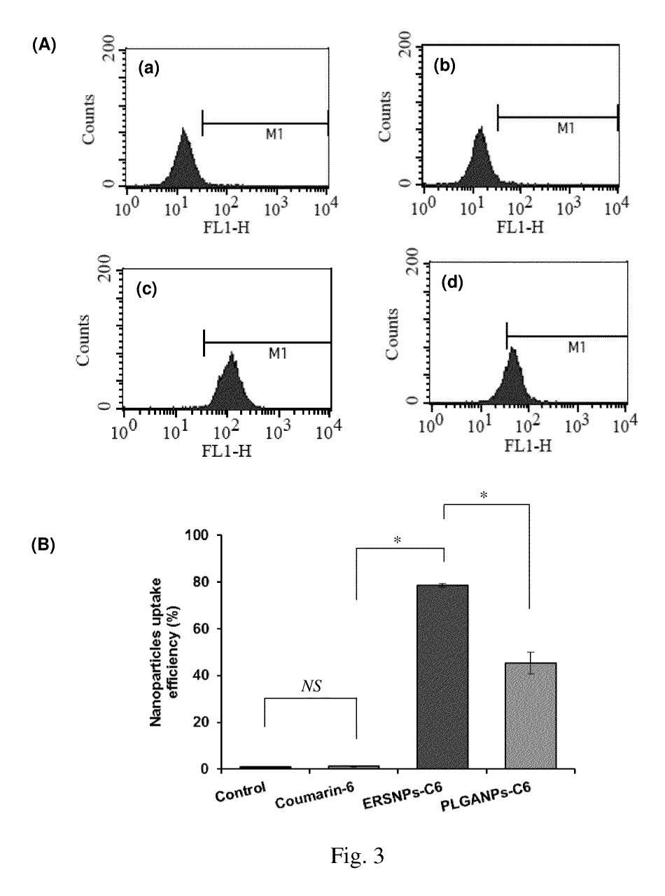

FIG. 3A depicts fluorescence intensity of cells incubated with HBSS, coumarin-6, ERSNPs-C6 and PLGANPs-C6 for 0.5 hours, and the corresponding nanoparticles uptake efficiency is depicted in FIG. 3B. The fluorescence intensity of cells incubated with free coumarin-6 (1.27±0.3%) was not significantly different from the control group (1.00±0.03%). It demonstrated that free coumarin-6 cannot be uptake by Caco-2 cells. However, a significant increase in the fluorescence intensity was observed after incubated with ERSNPs-C6 (78.39±0.76%) and PLGANPs-C6 (45.25±4.57%) (p<0.05). The Caco-2 cell monolayer incubated with ERSNPs-C6 and PLGANPs-C6 for 1 hour further increased cellular uptake efficiency to 98.67±3.27% and 79.25±4.50% (data not shown). The positively charged ERSNPs-C6 enhanced the cellular uptake more significantly than negatively charged PLGANPs-C6 (p<0.05).

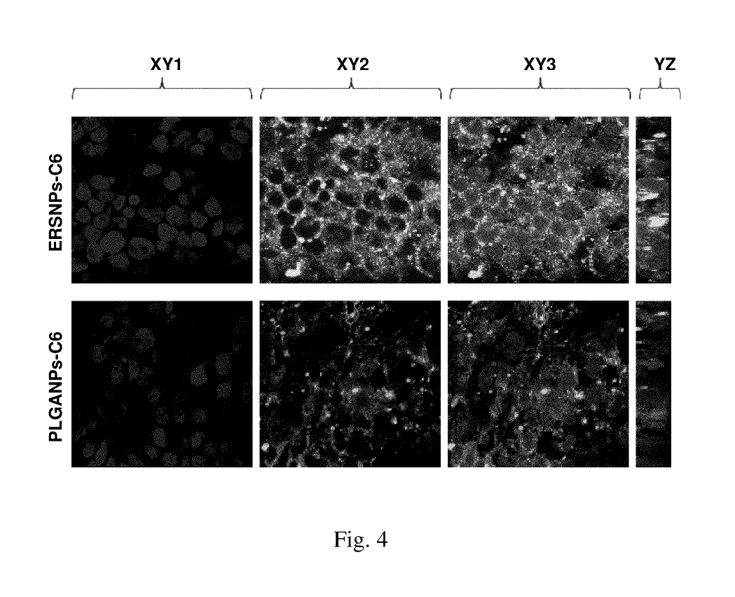

FIG. 4 depicts a confocal microscopic images of Caco-2 cell monolayers after incubated with ERSNPs-C6 and PLGANPs-C6 for 0.5 hours, wherein darker gray denotes ERSNPs-C6/PLGANPs-C6 and lighter gray denotes nuclei; XY1, XY2, and XY3 represent the images of FITC filter, RITC filter and FITC-RITC filter overlay; and Optical sections of xy plane with yz projections show the internalized nanoparticles (YZ). The strong green fluorescence in the cytoplasm indicates that the nanoparticles were internalized and localized in the cells after cellular uptake. It was further confirmed by three-dimensional analysis by reconstruction of z-axis of the confocal images of the cells after incubated with both kinds of nanoparticles where the fluorescent signals were clearly appeared inside the cells (YZ). This observation assured the internalization of the nanoparticles in Caco-2 cells.

### Example 4.4

### Biodistribution of Nanoparticles in Stomach

Male Wistar rats (250-300 g) were used in this study. They were obtained from National Laboratory Animal Center, Taipei, Taiwan. All animal experiments were carried out in accordance with the regulations of the Institutional Animal Care and Use Committee (IACUC) (National Taiwan University College of Medicine and College of Public Health, Taipei, Taiwan) and the animal experiment was in accordance with “Guide for the Care and Use of Laboratory Animals” published by the National Institute of Health.

Rats were fasted but allowed free access to water over night. Gastric ulcer was induced by oral administration of absolute ethanol (5 mL/kg). The ulcer induced rats were divided into 3 groups (1 control and 2 treatment) and each group consisted of 4 rats. Each treatment group received a hard gelatin capsule (#9, Torpac Inc., NJ, USA) filled with ERSNPs-C6 or PLGANPs-C6 (100 mg nanoparticles/kg) mixed with sodium bicarbonate (20 mg/kg) (ERSNPs-C6-NaHCO₃ and PLGANPs-C6-NaHCO₃) and the control group received saline solution. The formulations were administered orally 1 hour after the administration of the ethanol. Rats were sacrificed after 4 hour of dose administration.

The stomach was opened longitudinally and rinsed with saline solution. The ulcerated regions and non-ulcerated regions of stomach tissue were cut and the freshly excised tissues were cryofixed by TissueTek® Compound. The molded tissue sample was sectioned using Cryostat (Leica CM3050 S, Leica Microsystems, Wetzlar, Germany and observed under a fluorescence microscope combined with a photomicrography digitally integrate system (Zeiss Axiophot 2, Carl Zeiss, Hamburg, Germany). In addition, sectioned stomach tissues were stained with hematoxylin and eosin stain (H-E stain) to show the morphology of healthy and ulcerated tissues.

For quantitative determination, freshly excised tissues were lyophilized in the dark. Acetone 5 mL was added to the tissue sample and sonicated for 15 min The tissue samples were centrifuged at 2000 rpm for 5 min and the supernatant was collected. The extraction procedure was repeated 3 times. Finally, the supernatant was diluted with acetone and analyzed by fluorescence spectrophotometer (F4500, Hitachi Ltd., Tokyo, Japan) at an excitation wavelength 430 nm and an emission wavelength 490 nm.

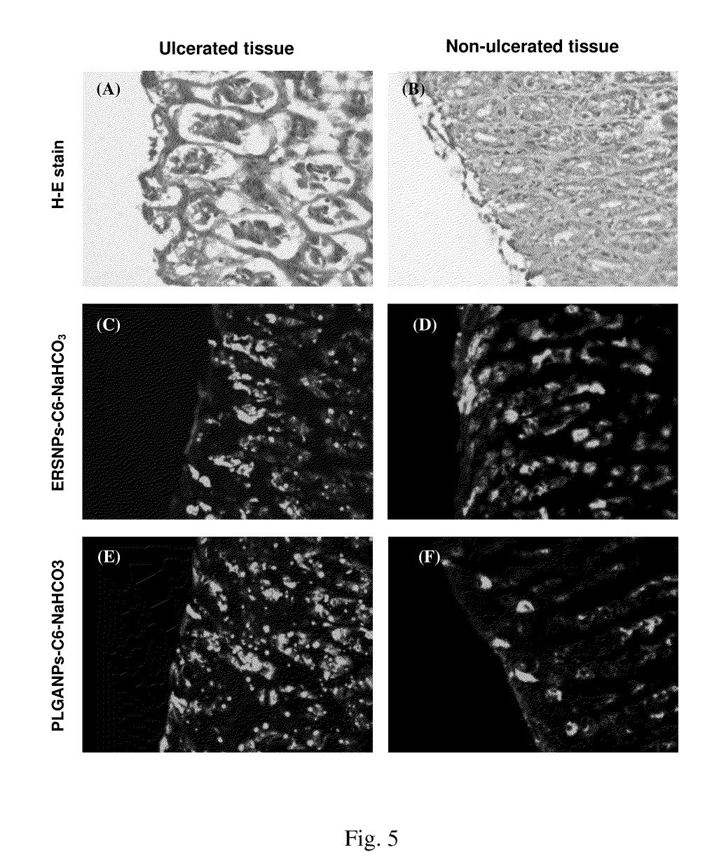

FIG. 5 depicts fluorescence microscopic images of ulcerated and non-ulcerated region of the stomach tissues after oral administration of ERSNPs-C6 and PLGANPs-C6 (100 mg/kg) mixed with sodium bicarbonate (20 mg/kg) (ERSNPs-C6-NaHCO₃ and PLGANPs-C6-NaHCO₃) to ulcer induced rats for 4 hours. FIG. 5A and FIG. 5B depicts hematoxylin-eosin (H-E) stained ulcerated and non-ulcerated regions of stomach tissues of ulcer induced rats before nanoparticles treatment. The nanoparticles were localized in both ulcerated (FIG. 5C and FIG. 5E) as well as non-ulcerated (FIG. 5D and FIG. 5F) regions of stomach tissues in ulcer induced rats after nanoparticles treatment.

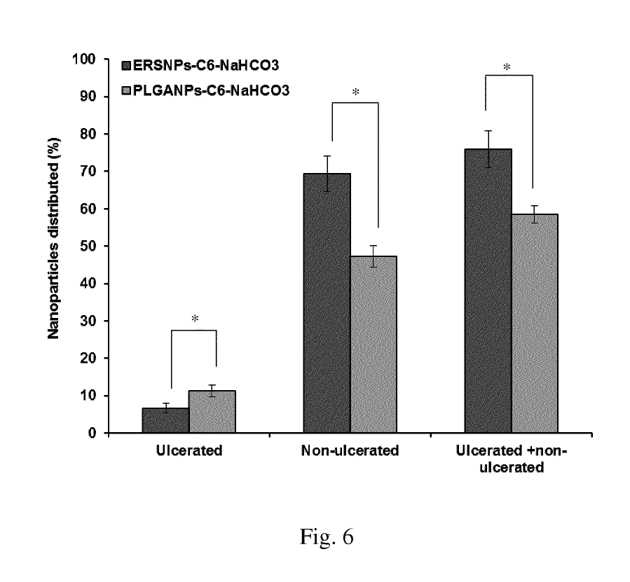

FIG. 6 depicts the quantitative determination of both nanoparticles in ulcerated stomach tissues. More ERSNPs-C6-NaHCO₃ (69.28±4.78%, FIG. 6D) were adhered than PLGANPs-C6-NaHCO₃ (47.21±2.89%, FIG. 6F) in the non-ulcerated region. On the other hand, more PLGANPs-C6-NaHCO₃ (11.23±1.59%, FIG. 6E) were adhered than ERSNPs-C6-NaHCO₃ (6.59±1.30%, FIG. 6C) in the ulcerated region while the total amount of ERSNPs-C6-NaHCO₃ deposited in both ulcerated and non-ulcerated regions (75.87±4.94%) was higher than PLGANPs-C6-NaHCO₃ in ulcerated and non-ulcerated regions (58.44±2.33%). The negatively charged PLGANPs-C6 exhibited high affinity towards positively charged ulcerated cell membranes, and therefore exhibited increased bioadhesion to the ulcerated region. Oppositely, positively charged ERSNPs-C6 showed high affinity towards negatively charged cell membrane, and therefore exhibited increased bioadhesion to non-ulcerated region. These results suggested that prepared nanoparticles have great potential for stomach-specific delivery of LPZ.

### Example 5

### Pharmacokinetic Study

Male Wistar rats (250-300 g) were used in this study. All procedures were examined by the IACUC as mentioned in Example 4.4.

Rats were fasted but allowed free access to water over night. The rats were divided into 3 different groups and each group consisted of 4 rats. A known commercial product RICH® (a capsule containing enteric coated pellets), or the hard gelatin capsule (#9, Torpac Inc., NJ, USA) filled with (i) ERSNPs-LPZ (5 mg LPZ/kg) and sodium bicarbonate (20 mg/kg) (ERSNPs-LPZ-NaHCO₃) or (ii) PLGANPs-LPZ (5 mg LPZ/kg) and sodium bicarbonate (20 mg/kg) (PLGANPs-LPZ-NaHCO₃) were orally administered to rats. Blood samples were collected from tail veins of rats prior to drug administration and at time intervals of 0.5, 1, 1.5, 2, 3, 4, 5, 6, 8, 10, 12 and 24 hours after dosing. The blood samples were centrifuged at 12,000 rpm for 10 min at 4° C. and the supernatant was stored at −80° C. until analysis.

The serum LPZ concentrations were determined by HPLC method. LPZ was extracted from the plasma samples by modifying liquid-liquid extraction method. Acetonitrile 400 μL was added into 100 μL plasma to precipitate proteins. The mixture was vortex for 60 s following by centrifugation at 12000 rpm for 10 min (Eppendorf centrifuge 5804R, Eppendorf Co. Ltd., NY, USA). The supernatant was collected, air dried, and reconstituted with 45 μL of the mobile phase of which 20 μL was injected into the HPLC system. The HPLC system (Jasco International Company Ltd., Tokyo, Japan) was consisted of a pump (PU-2089) and a photo diode array detector (PDA, MD-2010) at 285 nm A reversed phase silica column (C-18, 4.6×250 mm, 5 μm, Phenomenex Inc., USA) was used. The mobile phase was comprised by water, acetonitrile and triethylamine in the volume ratio of 50:50:0.1 (pH 7) with a flow rate of 1 mL/min.

The HPLC analytical method was validated prior to sample analysis.

The LPZ solutions (in mobile phase) in the concentration range of 10-1000 ng/mL were spiked with blank plasma and follow the same extraction method as mentioned above. Each reconstituted sample was analyzed by HPLC method. The standard curves were found to be linear with the coefficients of determination (R²) greater than 0.9979. The lower limit of quantification was 10 ng/mL. The accuracy was in the range of 93.33%-109.00%, and the precision was in the range of 0.66-6.91%. The pharmacokinetic parameters were obtained from the plasma LPZ concentration-time data based on a noncompartmental pharmacokinetic analysis model (WinNonlin software, version 5.3, Pharsight Corporation, CA, USA).

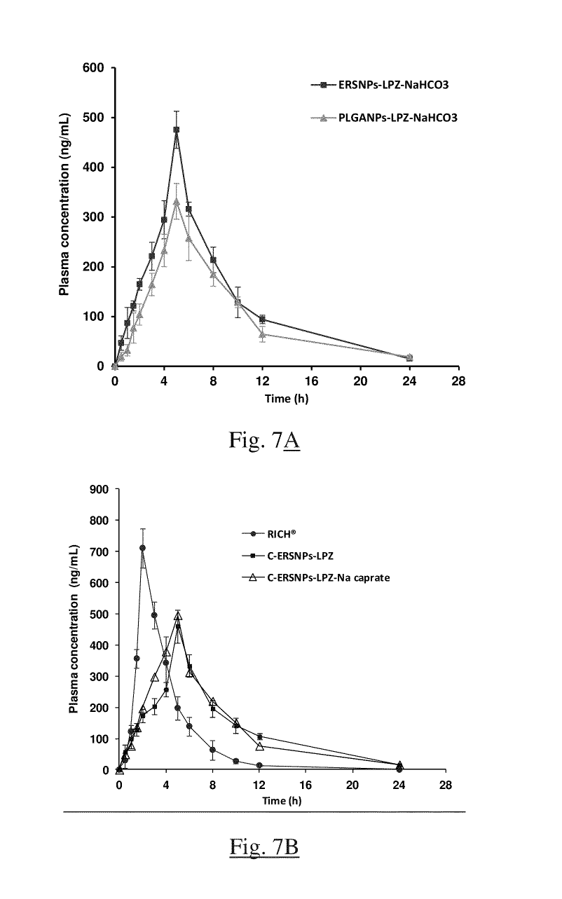

FIG. 7 depicts plasma LPZ concentration versus time profiles after oral administration of ERSNPs-LPZ-NaHCO₃, PLGANPs-LPZ-NaHCO₃ (5 mg LPZ/kg), or the known commercial product RICH® in ulcer induced male Wistar rats.

The AUC_{0-∞} values of ERSNPs-LPZ-NaHCO₃ and PLGANPs-LPZ-NaHCO₃ were 3253.63±129.39 and 2579.74±254.85 ng•h/mL, respectively. The AUC_{0-∞} of ERSNPs-LPZ-NaHCO₃ was higher than that of PLGANPs-LPZ-NaHCO₃ (p<0.05). The mean Cₘₐₓ values of ERSNPs-LPZ-NaHCO₃ and PLGANPs-LPZ-NaHCO₃ were 475.34±37.47 and 331.7±35.96 ng/mL, respectively, with the same Tₘₐₓ values 5 hours, and the corresponding T_{1/2} values were 4.60±0.45 and 4.71±0.41 hours. The V_{d}/F and CL/F of ERSNPs-LPZ-NaHCO₃ were lower than PLGANPs-LPZ-NaHCO₃. This suggest that the bioavailability of LPZ was higher in ERSNPs-LPZ-NaHCO₃ than in PLGANPs-LPZ-NaHCO₃. This was because the positive charge of ERSNPs-LPZ-NaHCO₃ had higher affinity towards negatively charged cell surface due to electrostatic interaction than negatively charged PLGANPs-LPZ-NaHCO₃.

Since the conventional drug formulation for treatment of acid related disorders is enteric coated, its sustainably released profile may be used to compare with the nanoparticulate dosage form of the present disclosure.

The AUC_{0-∞} value was 2260.37±272.90 ng•h/mL for RICH®, which was lower than the AUC_{0-∞} values of ERSNPs-LPZ-NaHCO₃ and PLGANPs-LPZ-NaHCO₃ (p<0.05). The relative bioavailabilities (BA_{R}) of ERSNPs-LPZ-NaHCO₃ and PLGANPs-LPZ-NaHCO₃ were 143.95% and 114.13% in comparison to RICH®. The Tₘₐₓ values were 5 hour for ERSNPs-LPZ-NaHCO₃ and PLGANPs-LPZ-NaHCO₃ while 2 hour for RICH®. The corresponding T₁₁₂ values of ERSNPs-LPZ-NaHCO₃, PLGANPs-LPZ-NaHCO₃ and RICH® were 4.60±0.45, 4.71±0.41 and 1.74±0.10 hour, respectively. As can be seen in FIG. 7, the nanoparticulate dosage form of the present disclosure not only improved the extent of drug absorption in terms of higher bioavailability of LPZ but also extended the retention time of LPZ in the blood circulation as compared to RICH®.

### Example 6

### Ulcer Healing Response

Male Wistar rats (250-300 g) were used in this study. All procedures were examined by the IACUC, as mentioned in Example 4.4.

Rats were fasted but allowed free access to water over night. The gastric ulcer was induced 1 hour after oral administration of absolute ethanol (5 mL/kg). The rats were divided into 3 groups, and each group was consisted 4 rats. Each group received saline solution 1 mL (control) or two different nanoparticle formulations as mentioned in Example 5. The formulations were administered orally 1 hour after the administration of the ethanol and multiple doses were administered once daily for 7 days. Rats were sacrificed after 24 hours of the last dose administered.

The stomachs were cut along with the greater curvature and the stomach mucosal surface was washed with saline solution (0.9% NaCl). The photographic images (Nikon E5000, Nikon Corporation, Tokyo, Japan) of stomach mucosal surface were taken and the total ulcerated area and mucosal area were measured using Axio Vision software (version 4.8, Carl Zeiss International, NY, USA). The ulcer index (UI) was calculated using Eq. (4).

$$Ulcer Index = \frac{Total ulcerated area \times 10}{Total mucosal area} \qquad ( 4 )$$

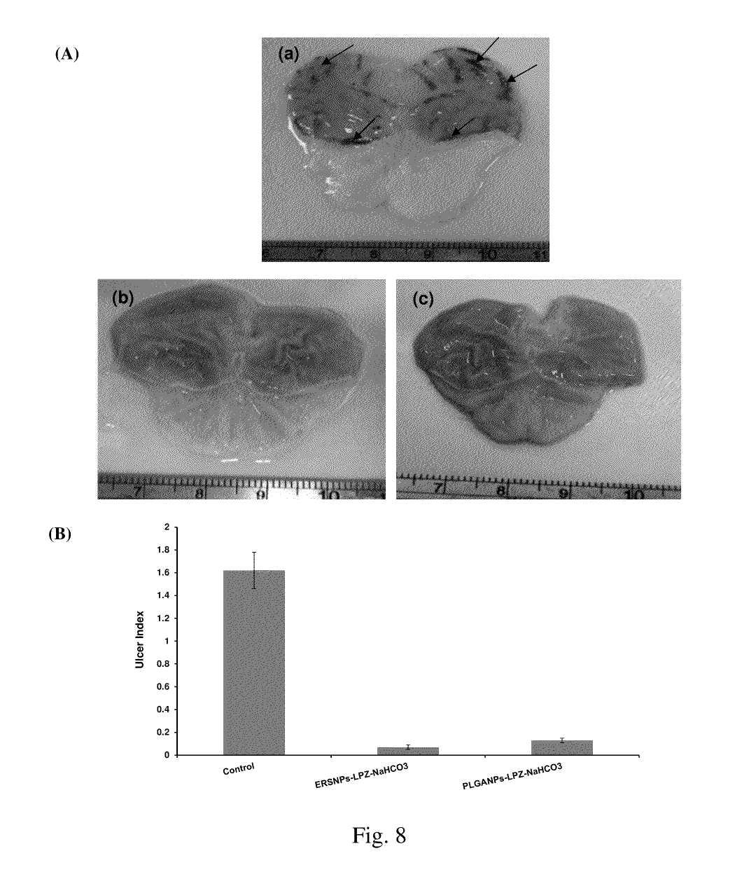

The healing promoting activity of prepared LPZ nanoparticles was further demonstrated in ulcerated rats. FIG. 8A shows the photographic images of stomachs in ulcer induced rats after oral administrations of saline solution, ERSNPs-LPZ-NaHCO₃ and PLGANPs-LPZ-NaHCO₃ for 7 days. The gastric ulcer indexes of the control group (saline solution), ERSNPs-LPZ-NaHCO₃ and PLGANPs-LPZ-NaHCO₃ were 1.62±0.16, 0.07±0.02 and 0.12±0.02, respectively (FIG. 8B). These results suggested that the induced gastric ulcer was healed gradually within one week after oral administration of LPZ nanoparticles (5 mg LPZ/kg/day), and the ulcer healing efficiency was about 95%. The ulcer healing efficiency of LPZ nanoparticles was efficient due to their sustained release property and prolonged in vivo absorption which can control the acid secretion for 24 h. The present invention successfully demonstrated that the prepared non-enteric LPZ nanoparticles exhibited a healing promoting action on pre-existing gastric ulcer in rats.

While the present invention is disclosed by reference to the preferred embodiments and examples detailed above, it is to be understood that these examples are intended in an illustrative rather than in a limiting sense. It is contemplated that modifications and combinations will readily occur to those skilled in the art, which modifications and combinations will be within the spirit of the invention and the scope to of the following claims and its equivalent systems and methods.

What is claimed is:

1. A non-enteric coated pharmaceutical composition having an enhanced bioavailability, consisting essentially of:
an acid-labile active ingredient, and
a biocompatible polymer;
wherein the acid-labile active ingredient is mixed with and trapped by the biocompatible polymer, and the acid-labile active ingredient is sustainably released from the biocompatible polymer;
wherein the non-enteric coated pharmaceutical composition is without an enteric coating film and is in a form of nanoparticle;
wherein the enhanced bioavailability of the non-enteric coated pharmaceutical composition is presented by the higher AUC_{0-∞} value compared with that of the same acid-labile active ingredient with enteric coating film.

2. (canceled)

3. The non-enteric coated pharmaceutical composition of claim 1, wherein the nanoparticle has an average diameter of about 100 nm to about 950 nm.

4. The non-enteric coated pharmaceutical composition of claim 1, wherein the nanoparticle has an average diameter of about 120 nm to about 300 nm with a polydispersity index less than 110.3.

5. The non-enteric coated pharmaceutical composition of claim 1, wherein the active ingredient is selected from the group consisting of omeprazole, lansoprazole, dexlansoprazole, esomeprazole, pantoprazole, minoprazole, pantoprazole and rabeprazole, penicillin salts, bacitracin, aureomycin, cephalosporins, chloromycetin, erythromycin, dihydrostreptomycin, streptomycin, novobiocin, polymyxin, subtilin, famotidine, progabide, clorazepate, deramciclane, pravastatin, milameline, digitalis glycosides, etoposide, quinapril, quinoxaline-2-carboxylic acid, sulphanilamide, beta carotene, cladribine, didanosine, amylase, lipase, protease, adrenalin, insulin, heparin, estrogens, cisapride, ranitidine, pancreatin, and cimetidine.

6. The non-enteric coated pharmaceutical composition of claim 1, wherein the active ingredient is for treating or preventing a stomach disorder.

7. The non-enteric coated pharmaceutical composition of claim 6, wherein the active ingredient is selected from the group consisting of antacids, H2 receptor antagonists, and proton pump inhibitors.

8. The non-enteric coated pharmaceutical composition of claim 7, wherein the active ingredient is selected from the group consisting of omeprazole, lansoprazole, dexlansoprazole, esomeprazole, pantoprazole, minoprazole, pantoprazole, and rabeprazole.

9. The non-enteric coated pharmaceutical composition of claim 1, wherein the biocompatible polymer is selected from the group consisting of poly(acrylic acid), polyacrylate, polycyanoacrylate, polyanhydride, polyamide, polyester, poly(orthoester), polyesteramide, polydihydropyran, poly(lactic acid), poly(glycolic acid), poly(lactic-co-glycolic acid), poly(ethylene glycol), polyvinyl alcohol (PVA), poly(sulfobetaine methacrylate) (PSBMA), polyhydroxyalkanoate (PHA), poly(hydroxyhexanoate), polyphosphazene, polypeptide, and a copolymer comprising monomers selected from a group consisting of ethyl acrylate, methyl methacrylate, and methacrylic acid.

10. The non-enteric coated pharmaceutical composition of claim 1, wherein the biocompatible polymer is a copolymer comprising monomers selected from a group consisting of ethyl acrylate, methyl methacrylate, and methacrylic acid.

11. The non-enteric coated pharmaceutical composition of claim 1, wherein the biocompatible polymer is poly(lactic-co-glycolic acid).

12. A method for treating or preventing a stomach disorder, comprising:
administering a non-enteric coated pharmaceutical composition to a patient subjected to said stomach disorder;
wherein the non-enteric coated pharmaceutical composition comprises an acid-labile active ingredient for treating or preventing said stomach disorder and a nanolized biocompatible polymer for mixing and trapping the acid-labile active ingredient.

13. The method of claim 12, wherein the pharmaceutical composition is a sustained release form with enhanced bioavailability.

14. The method of claim 12, wherein the pharmaceutical composition has a controlled release of about 24 hours per dosage.

15. The method of claim 12, wherein the pharmaceutical composition is in a form of a nanoparticle.

16. The method of claim 12, wherein the active ingredient is selected from the group comprising antacids, H2 receptor antagonists, and proton pump inhibitors.

17. The method of claim 16, wherein the active ingredient is selected from the group comprising omeprazole, lansoprazole, dexlansoprazole, esomeprazole, pantoprazole, minoprazole, pantoprazole, and rabeprazole.

18. The method of claim 12, wherein the biocompatible polymer is poly(lactic co-glycolic acid) or a copolymer comprising monomers selected from a group comprising ethyl acrylate, methyl methacrylate, and methacrylic acid, or a combination thereof.

19. The method of claim 12, wherein the stomach disorder comprises peptic ulcer and gastro-esophageal reflux disease (GERD).
